(19) United States
(12) Patent Application Publication
(10) Pub. No.: US 2015/0157293 A1
(43) Pub. Date: Jun. 11, 2015

(54) ULTRASOUND PROBE

(71) Applicants: Kabushiki Kaisha Toshiba, Minato-ku (JP); Toshiba Medical Systems Corporation, Otawara-shi (JP)

(72) Inventors: Koichi SHIBAMOTO, Nasushiobara (JP); Hideki Kosaku, Nasushiobara (JP); Takashi Kubota, Otawara (JP); Takashi Takeuchi, Otawara (JP); Minoru Aoki, Yaita (JP)

(73) Assignees: Kabushiki Kaisha Toshiba, Minato-ku (JP); Toshiba Medical Systems Corporation, Otawara-shi (JP)

(21) Appl. No.: 14/559,270

(22) Filed: Dec. 3, 2014

(30) Foreign Application Priority Data
Dec. 9, 2013 (JP) 2013-253793

(51) Int. Cl. A61B 8/00; A61B 8/12

(52) U.S. Cl. CPC A61B 8/4455; A61B 8/4488; A61B 8/12

(57) ABSTRACT

The ultrasound probe insertable into a body cavity of a subject has a clamped member including a plurality of pairs of clamped surfaces capable of being clamped by a forcep. The clamped member is formed so as to protrude from a surface which is the opposite side of the surface from where ultrasound waves are emitted, among the surfaces of the probe head.

### CROSS-REFERENCE TO RELATED APPLICATION

This application is based upon and claims the benefit of priority from Japanese Patent Application No. 2013-253793, filed Dec. 9, 2013; the entire contents of which are incorporated herein by reference.

### FIELD

The embodiments of the present invention relate to ultrasound probes.

### BACKGROUND

Ultrasound diagnosis apparatuses transmit ultrasound waves into a subject using an ultrasound probe, and receive reflected waves from the subject to obtain biological information of the subject. The obtained biological information is used to generate an ultrasound image representing a shape or function of the subject.

For example, when an ultrasound diagnosis apparatus is used in the operation under a celoscope, an ultrasound probe and a forcep are inserted into a body cavity through a trocar (cylindrical member) formed at a body wall of the subject. An operator operates the ultrasound probe while clamping the ultrasound probe with the forcep. The operator moves the ultrasound probe by operating the ultrasound probe.

### BRIEF DESCRIPTION OF THE DRAWINGS

FIG. 1 is a schematic diagram illustrating an outline of an ultrasound probe according to an embodiment.

FIG. 2 is a schematic diagram illustrating an outline of the ultrasound probe according to the embodiment.

FIG. 3 is a schematic diagram illustrating an outline of a use state of the ultrasound probe according to the embodiment.

FIG. 4 is a schematic diagram illustrating an outline of a use state of the ultrasound probe according to the embodiment.

FIG. 5 is a schematic diagram illustrating an outline of the ultrasound probe according to the embodiment.

FIG. 6 is a schematic diagram illustrating an outline of the ultrasound probe according to the embodiment.

FIG. 7 is a schematic diagram illustrating an outline of the ultrasound probe according to the embodiment.

FIG. 8 is a schematic diagram illustrating an outline of the ultrasound probe according to the embodiment.

FIG. 9 is a schematic diagram illustrating an outline of the ultrasound probe according to the embodiment.

FIG. 10 is a schematic diagram illustrating an outline of an ultrasound probe according to an embodiment.

FIG. 11 is a schematic diagram illustrating an outline of the ultrasound probe according to the embodiment.

FIG. 12 is a schematic diagram illustrating an outline of the ultrasound probe according to the embodiment.

FIG. 13 is a schematic diagram illustrating an outline of the ultrasound probe according to the embodiment.

FIG. 14 is a schematic diagram illustrating an outline of the ultrasound probe according to the embodiment.

FIG. 15 is a schematic diagram illustrating an outline of the ultrasound probe according to the embodiment.

FIG. 16 is a schematic diagram illustrating an outline of the ultrasound probe according to the embodiment.

FIG. 17 is a schematic diagram illustrating an outline of the ultrasound probe according to the embodiment.

### DETAILED DESCRIPTION

An ultrasound probe as the embodiment is the ultrasound probe insertable into a body cavity of a subject. The ultrasound probe has a clamped member including a plurality of pairs of clamped surfaces capable of being clamped by a forcep. The clamped member is formed so as to protrude from a surface which is the opposite side of the surface from where ultrasound waves are emitted, among the surfaces of the probe head.

### First Embodiment

### [Configuration]

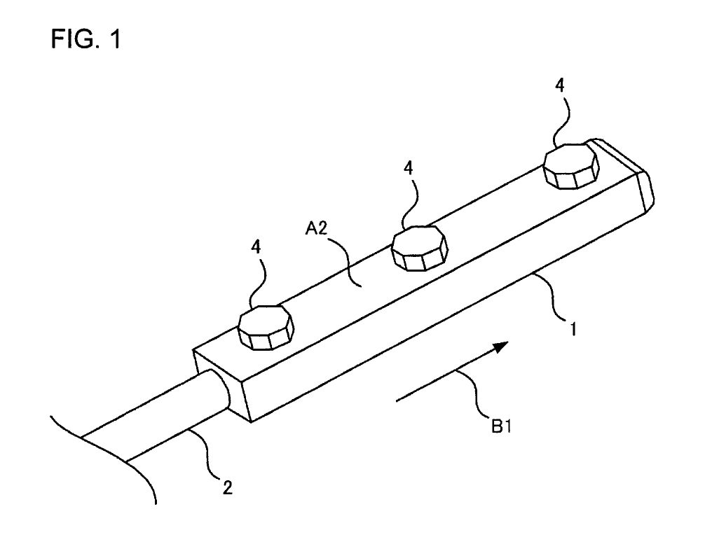

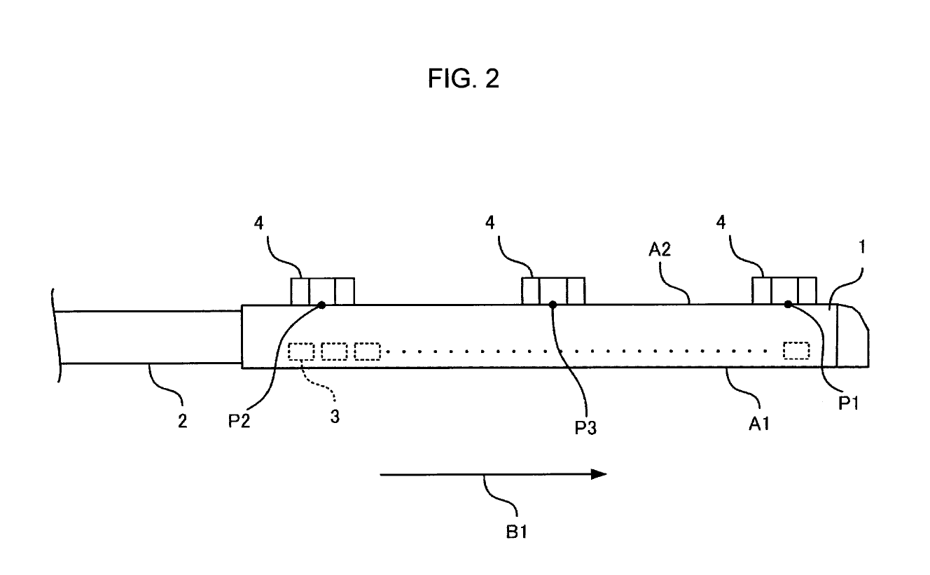

FIG. 1 is a perspective diagram schematically illustrating an ultrasound probe of a first embodiment. FIG. 2 is a side-elevation diagram schematically illustrating the ultrasound probe of the embodiment.

The ultrasound probe is configured to be operatable by a forcep 5. Hereinafter, the configuration of the ultrasound probe is described.

The forcep 5 has an opening/closing member configured to be openable/closeable. The forcep 5 opens/closes the opening/closing member through the operation by the operator to clamp or release clamped members 4 (later described).

### (Ultrasound Probe)

The ultrasound probe transmits ultrasound waves to a subject, and receives reflected waves from the subject. The ultrasound probe has a probe head 1 and a cable 2.

### (Probe Head 1)

The probe head 1 is insertable to a subject body cavity. The housing of the probe head 1 is made from materials insertable into a body cavity. The probe head 1 has a plurality of ultrasound transducer 3 in the housing. The ultrasound transducers 3 generate ultrasound waves by being vibrated based on control signals from an ultrasound diagnosis apparatus. The generated ultrasound waves are transmitted to the subject. The ultrasound transducers 3 receive reflected waves from the subject, and output echo signals based on the received reflected waves to the ultrasound diagnosis apparatus.

The probe head 1 transmits ultrasound waves emitted from a predetermined surface to the subject, and receives reflected waves from the surface. For example, in the housing of the probe head 1, the ultrasound transducers 3 are arranged in the vicinity of a surface A1 (predetermined surface) from where the ultrasound waves are emitted. In the housing of the probe head 1, a backing material (not shown) for absorbing the ultrasound waves is provided between a surface A2 which is the opposite side surface of the surface A1 from where the ultrasound waves are emitted and the ultrasound transducers 3. The material for the housing of the probe head 1, the material for the ultrasound transducers 3, and the material for the backing material are appropriately selected from typical materials. The probe head 1 has the clamped members 4.

### (Clamped Member 4)

The clamped members 4 are formed so as to protrude from a surface of the probe head 1. For example, the surface of the probe head 1 is an outer surface of the housing of the probe head 1. Hereinafter, the surface of the probe head 1 is assumed to be the outer surface of the housing of the probe head 1. The clamped members 4 are formed so as to protrude from the surface different from the surface A1 from where ultrasound waves are emitted, among the surfaces of the probe head 1. For example, the surface A1 from where ultrasound waves are emitted is a surface in the vicinity of the arrayed ultrasound transducers 3. The clamped members 4 of the embodiment are formed so as to protrude from, as the surface different from the surface A, the surface A2 which is the opposite side of the surface from where ultrasound waves are emitted.

The clamped members 4 are formed so as to protrude from a plurality of positions on the surface of the probe head 1. For example, the clamped member 4 are formed so as to protrude from a plurality of positions arrayed in a longitudinal direction B1 of the probe head 1. FIG. 2 illustrates a state in which the clamped members 4 are formed so as to protrude from a tip end side position P1, a base end side position P2, and a central position P3 as a plurality of positions. The clamped member 4 includes a plurality of pairs of clamped surfaces. The pair of clamped surfaces is able to be clamped by the forcep 5. The tip end side position P1, the base end side position P2, and the central position P3 may be arranged in the same direction as the direction the ultrasound transducers 3 are arrayed (longitudinal direction B1). The plurality of clamped members 4 is formed so as to protrude from those positions. In other words, the plurality of clamped members 4 is arranged in the same direction as the direction the ultrasound transducers 3 are arrayed.

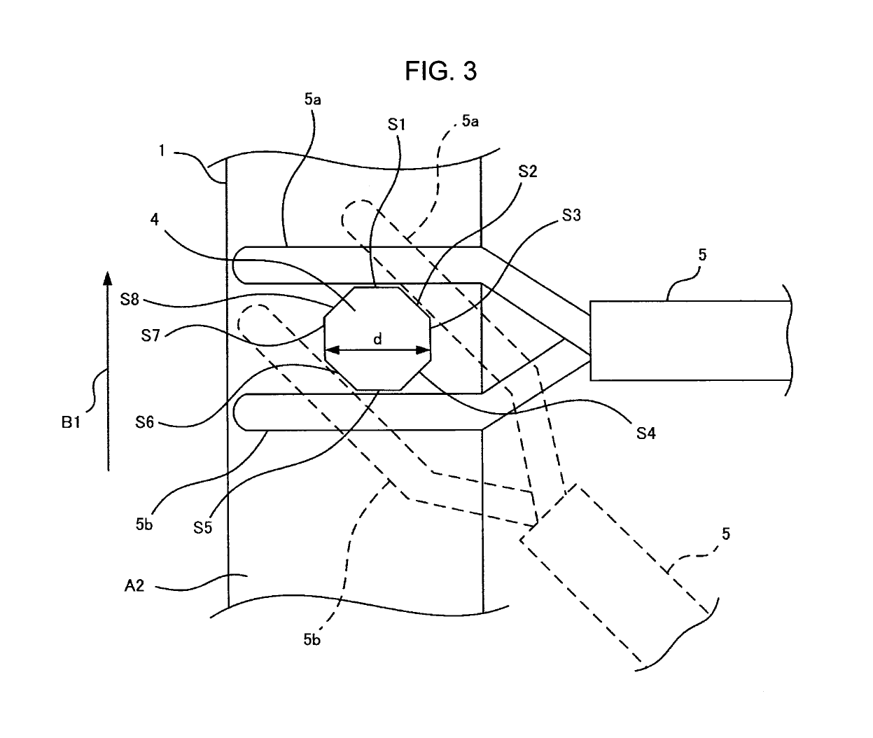

FIG. 3 is a schematic diagram illustrating a state (use state) in which the clamped member 4 is being clamped by the forcep 5. A pair of clamped surfaces is configured with two parallel surfaces. The clamped member 4 is formed in a polygonal pillar shape having an odd number of apexes and a polygonal bottom surface by including a plurality of pairs of clamped surfaces. The opposite sides of the polygon are parallel. Thus, the opposite sides of the polygonal pillar are parallel. The side surfaces of the polygonal pillar correspond to the clamped surfaces.

By forming the clamped member 4 into such the polygonal pillar shape, any one pair among the plurality of clamped surface pairs can be clamped. Thus, the operator can operate the ultrasound probe while clamping a desired pair of clamped surfaces using the forcep 5. The operator can change the angle of the probe head by shifting the clamped member 4 formed in the polygonal pillar shape by changing the pair of clamped surfaces to be clamped by the forcep 5 from one to the other in a different direction.

In the example shown in FIG. 3, 4 pairs of clamped surfaces (a pair having a clamped surface S1 and a clamped surface S5, a pair having a clamped surface S2 and a clamped surface S6, a pair having a clamped surface S3 and a clamped surface S7, and a pair having a clamped surface S4 and a clamped surface S8) are formed. Further, FIG. 3 illustrates a state in which the forcep 5 having a pair of clamping pieces 5a and 5b clamps the pair having the clamped surface S1 and the clamped surface S5 in a solid line, and a state in which the forcep 5 clamps the pair having the clamped surface S2 and the clamped surface S6 in a broken line. The clamped member 4 has the four pairs of clamped surfaces so as to be formed in an octagonal pillar shape. Any one of the clamped surface pairs of the clamped member 4 can be clamped by the forcep 5. In this example, there are four clamped surface pairs; the clamped member 4 can be clamped from any of four directions. A distance d between opposed surfaces of the clamped member 4 can be appropriately designed according to typical forceps.

### (Cable 2)

The cable 2 electrically connects the probe head 1 and the ultrasound diagnosis apparatus. For example, the cable 2 is connected to the base end side of the probe head 1. A part of the probe head 1 side of the cable 2 is inserted into the body cavity. The control signals from the ultrasound diagnosis apparatus are transmitted to the ultrasound transducers 3 through the cable 2. The echo signals from the ultrasound transducers 3 are transmitted to the ultrasound diagnosis apparatus through the cable 2. The cable 2 has a flexibility. During the operation, the cable 2 appropriately bends by following the position of the probe head 1.

### [Usage Example]

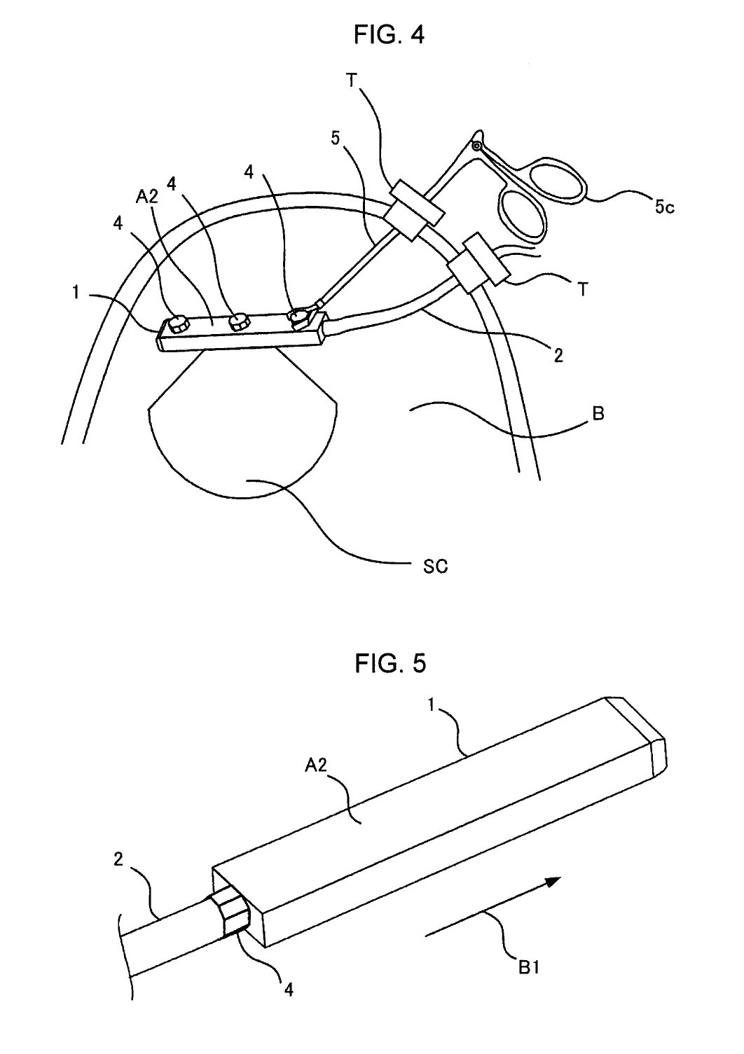

FIG. 4 is a schematic diagram illustrating a state (use state) in which the ultrasound probe of the embodiment is used in a body cavity B. The probe head 1 and the forcep 5 are inserted into the body cavity B through a trocar T arranged at the body wall of the subject. The operator uses the forcep 5 to operate the ultrasound probe inserted into the body cavity B. At this time, the operator clamps any one of the plurality of clamped surface pairs with the forcep 5 by operating a forcep operating member 5c. The operator can control the angle of the ultrasound probe by shifting the pair of clamped surfaces from one to another in accordance with the situation of the operation. Further, the operator can shift from the present clamped member 4 to the other clamped member at the different position. Thus, the operator can easily transfer the position of the ultrasound probe and control the angle thereof within a limited space, such as the body cavity B. Changing of the angle of the ultrasound probe performed by clamping the clamped member 4 with the forcep 5 corresponds to changing of a scanning surface SC of the ultrasound waves, by arraying the clamped member 4 in the same direction as the direction the ultrasound transducers 3. Thus, the operability of the ultrasound probe can be further improved.

### [Effect]

An effect of the ultrasound probe of the embodiment is now described. The ultrasound probe of the embodiment is the ultrasound probe having the probe head 1 which can be inserted into the body cavity of the subject, and the clamped members 4, each including a plurality pairs of clamped surfaces which can be clamped by the forcep 5, are formed so as to protrude from a surface of the probe head 1. There are four pairs of the clamped surfaces. The clamped members 4 are formed so as to protrude from a plurality of positions on a surface of the probe head 1. The clamped members 4 are formed so as to protrude from a surface different from the surface from where ultrasound waves are emitted, among the surfaces of the probe head 1. Thus, the ultrasound probe which can improve the operability by the forcep 5 can be provided.

### Modified Example 1 of the First Embodiment

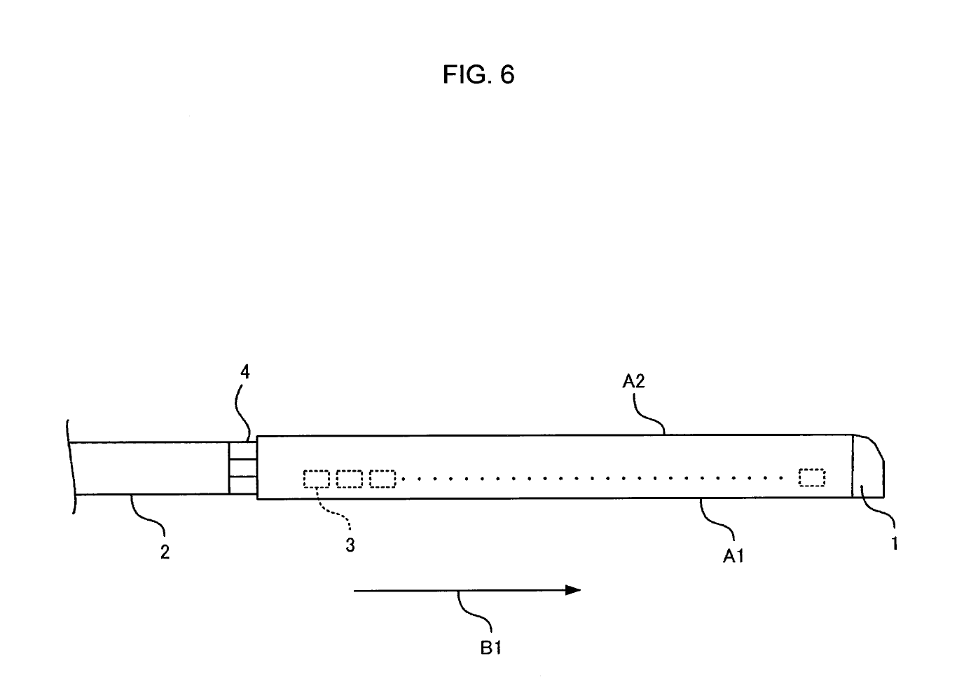

FIG. 5 is a perspective diagram schematically illustrating an ultrasound probe according to a modified example 1 of the first embodiment. FIG. 6 is a side-elevation diagram schematically illustrating the ultrasound probe according to the modified example. The position where the clamped member 4 of the ultrasound probe of the modified example is formed is different from that of the first embodiment. Hereinafter, matters which are different from that of the first embodiment are mainly described.

The clamped member 4 is formed so as to protrude from the surface to which the cable 2 is connected in the probe head 1. At this time, the cable 2 is inserted into the inside of the probe head 1 through the tip end side of the clamped member 4 and the base end side of the clamped member 4. The operator operates the ultrasound probe while clamping the clamped member 4 using the forcep 5. Even when the clamped member 4 is formed at the position illustrated in the modified example, the operability by the forcep 5 can be improved.

### Modified Example 2 of the First Embodiment

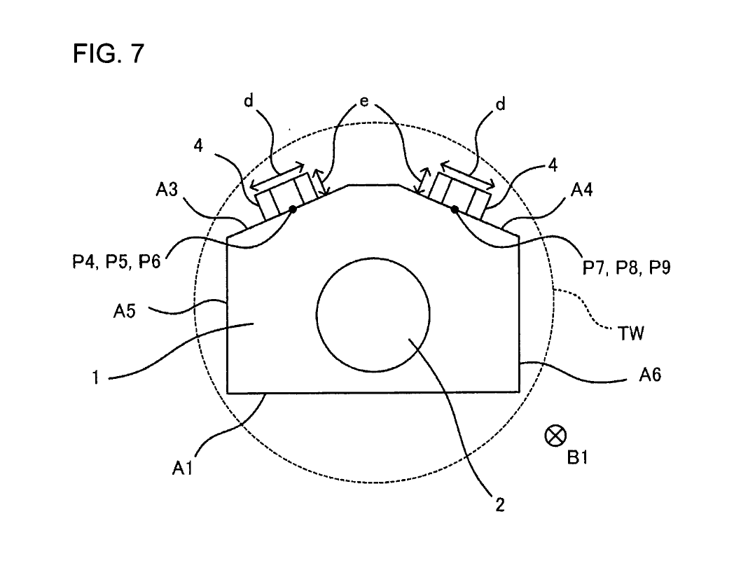

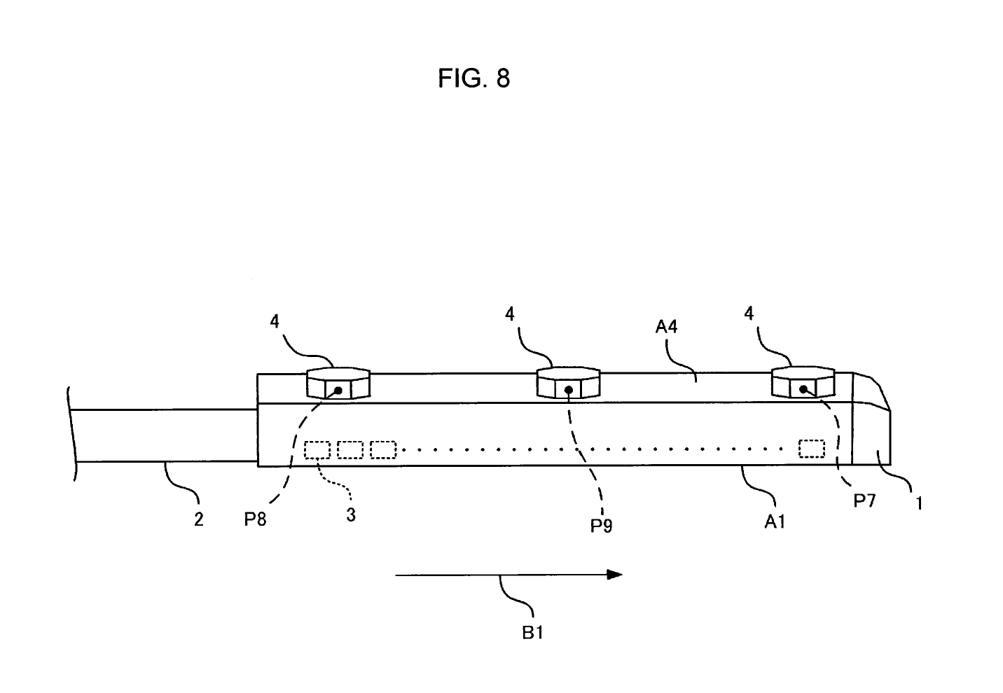

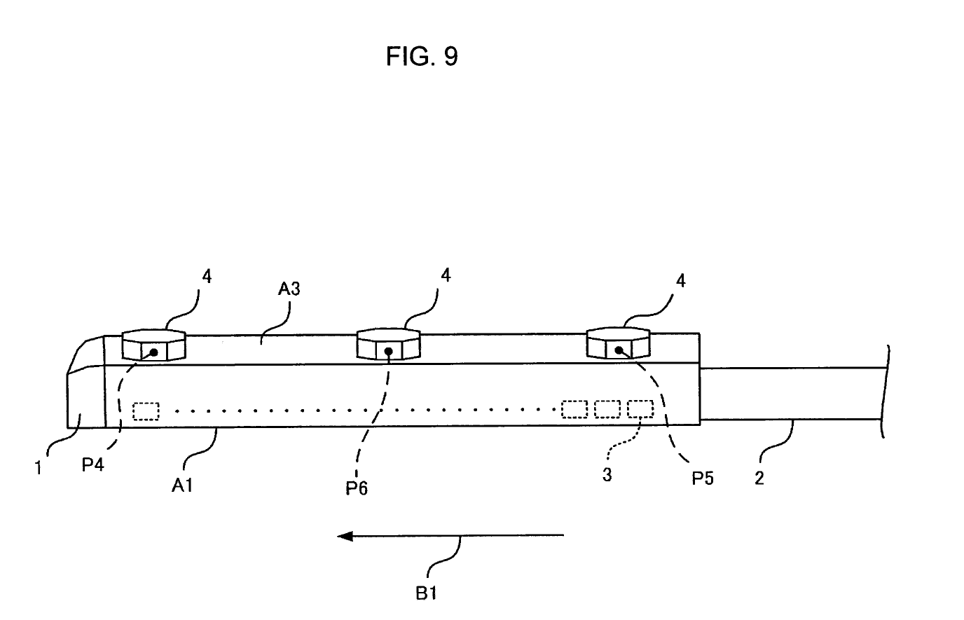

FIG. 7 is a projection diagram illustrating a state of the ultrasound probe according to a modified example 2 of the first embodiment seen from the base end side (cable 2 side). FIG. 8 and FIG. 9 are side-elevation diagrams schematically illustrating the ultrasound probe according to the modified example. In the ultrasound probe of the modified example, the shape of the housing of the probe head 1 and the positions where the clamped members 4 are formed are different from that of the first embodiment. Hereinafter, matters which are different from that of the first embodiment are mainly described.

The housing of the probe head 1 is formed with an inclined surface A3 and an inclined surface A4 provided by inclining to the surface A1 from where ultrasound waves are emitted. The clamped members 4 are formed so as to protrude from a plurality of positions on a surface different from the surface A1 from where ultrasound waves are emitted, among the surfaces of the probe head 1. The clamped members 4 of the modified example are formed so as to protrude from each of the inclined surface A3 and the inclined surface A4 which are different from the surface A1 from where ultrasound waves are emitted.

For example, in the inclined surface A3, the clamped members 4 are formed so as to protrude from a tip end side position P4, a base end side position P5, and a central position P6. Further, in the inclined surface A4, the clamped members 4 are formed so as to protrude from a tip end side position P7, a base end side position P8, and a central position P9. A distance d between opposed surfaces of the clamped member 4 and a protruding height e of the clamped member 4 are appropriately designed in dimensions which can be inserted into an inner wall TW of the trocar T. Similarly, the clamped members 4 may be formed on either one or both of a side surface A5 and a side surface A6 of the probe head 1.

There is a wide range of contents of movement of positions and control of angles for the ultrasound probe as for the operation performed while the ultrasound probe is being inserted into the body cavity B, depending on the purposes of the operation or subject parts. According to the modified example in which the probe head 1 is formed with the inclined surface A3 and the inclined surface A4, and the clamped members 4 are formed at the positions (P4 to P9), the operability by the forcep 5 can be improved since the ultrasound probe can be applicable for a wider range of contents of the operation.

### Second Embodiment

### [Configuration]

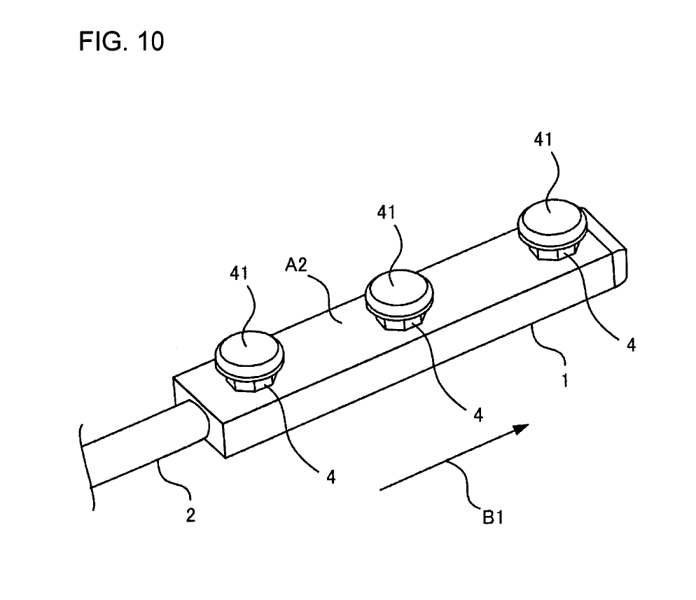

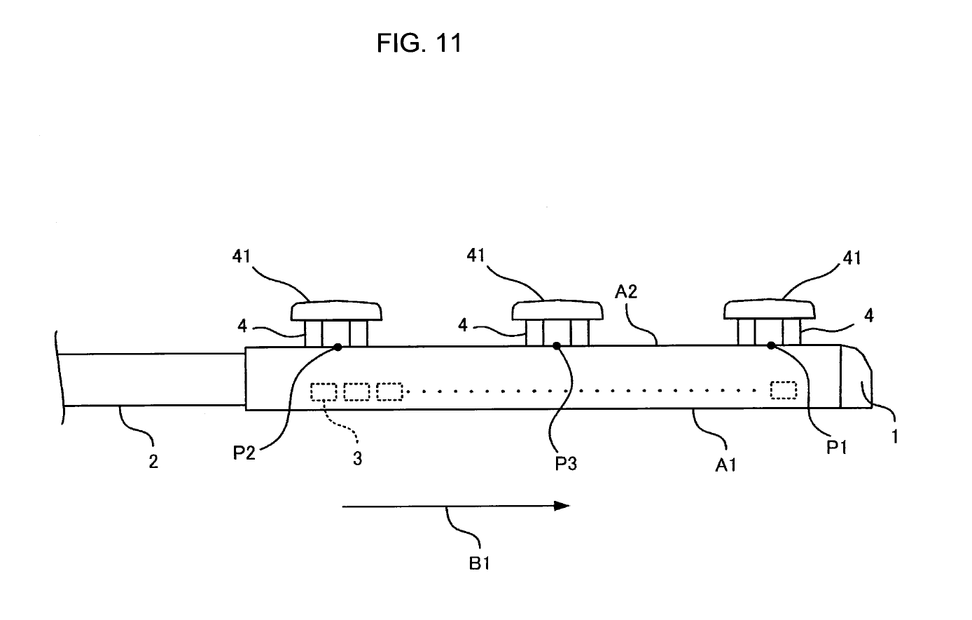

FIG. 10 is a perspective diagram schematically illustrating an ultrasound probe of a second embodiment. FIG. 11 is a side-elevation diagram schematically illustrating the ultrasound probe of the embodiment. The shape of the clamped member 4 of the ultrasound probe of the embodiment is different from that of the first embodiment. Hereinafter, matters which are different from that of the first embodiment are mainly described.

The clamped member 4 is formed with a step member 41 which makes the width of the tip end side wider than that of the base end side. Here, the base end side is the probe head 1 side of the clamped member 4. The tip end side is the opposite side of the base end side of the clamped member 4. The clamped members 4 are respectively formed so as to protrude from the tip end side position P1, the base end side position P2, and the central position P3 of the probe head 1. In the same manner as in the first embodiment, the tip end side position P1, the base end side position P2, and the central position P3 may be arranged in the direction same as the direction the ultrasound transducers 3 are arrayed.

In the same manner as in the first embodiment, the base end side of the clamped member 4 is formed in a polygonal pillar shape having a plurality of clamped surface pairs. Thus, the base end side is formed to be able to be clamped by the forcep 5. The part formed in the polygonal pillar shape is called a base end member. In this example, in the base end member, the shape of the cross-section perpendicular to the protruding direction of the clamped member 4 is assumed to be a regular octagon.

The tip end side of the clamped member 4 is formed with the step member 41 having a width wider than that of the base end side. Here, the width is assumed to be a dimension of the clamped member 4 in the direction perpendicular to the protruding direction.

The diameter of the circle which is the cross-sectional shape of the step member 41 is set longer than the length of the diagonal lines of the regular octagon which is the cross-sectional shape of the base end member. Thus, the width of the step member 41 is set longer than the width of the base end member. For example, when the forcep 5 clamping the base end side is being slipped off towards the tip end side, the forcep 5 is caught on the step member 41. Thus, the step member 41 prevents the forcep 5 from being slipped off from the base end side to the tip end side.

### [Effect]

An effect of the ultrasound probe of the embodiment is now described. The clamped member 4 of the ultrasound probe of the embodiment is formed with the step 41 which makes the width of the tip end side wider than that of the base end side. Thus, it is prevented that the forcep 5 is slipped off from the clamped member 4, and the ultrasound probe which can improve the operability by the forcep 5 can be provided.

### Modified Example 1 of the Second Embodiment

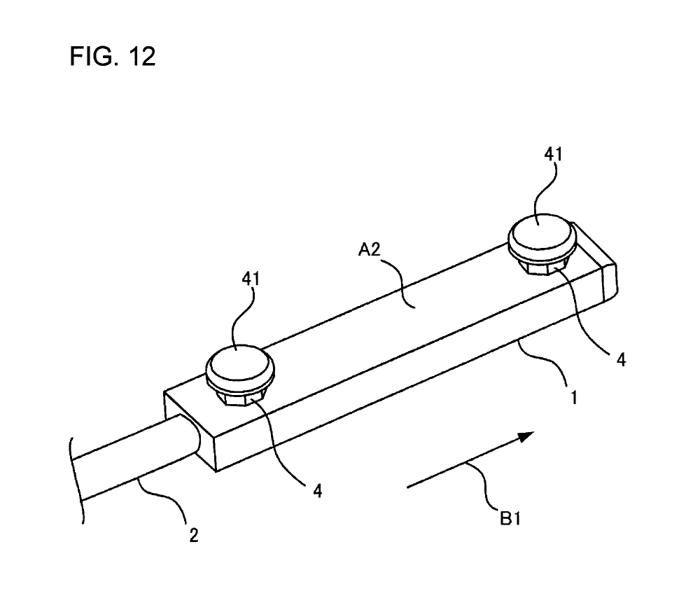

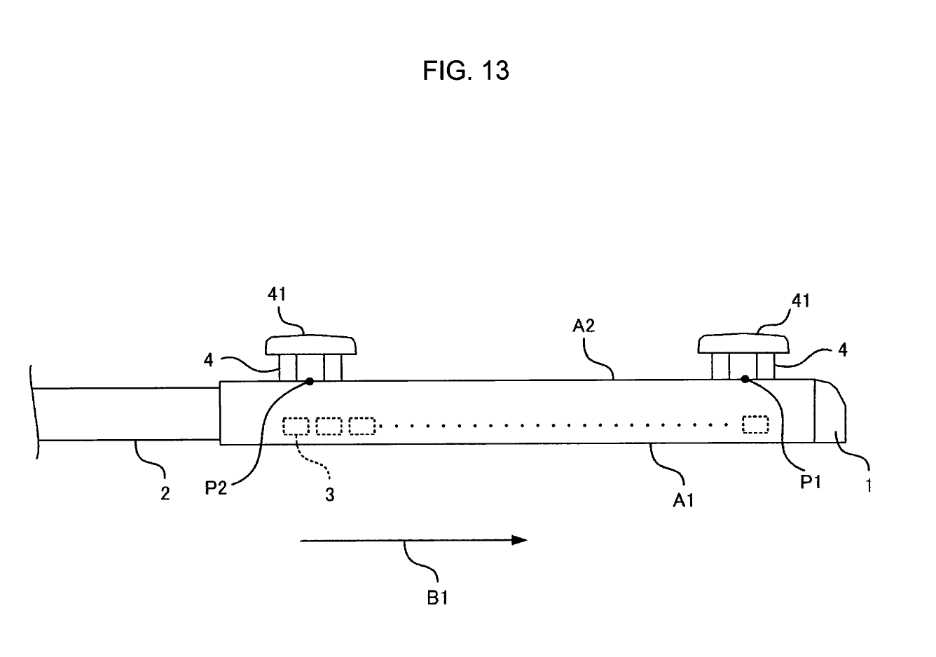

FIG. 12 is a perspective diagram schematically illustrating an ultrasound probe according to a modified example 1 of the second embodiment. FIG. 13 is a side-elevation diagram schematically illustrating the ultrasound probe according to the modified example. The positions of the clamped members 4 of the ultrasound probe of the modified example are different from that of the second embodiment. Hereinafter, matters which are different from that of the second embodiment are mainly described.

The clamped members 4 are respectively formed so as to protrude from the tip end side position P1 and the base end side position P2 of the probe head 1. For example, the operator clamps the clamped member 4 protruded from the tip end side position P1 with one of the forceps 5, and clamps the clamped member 4 protruded from the base end side position P2 with the other forcep 5. In this way, in the ultrasound probe, each of the clamped members 4 can be clamped by two forceps 5, respectively. Thus, the ultrasound probe can be stably clamped in the body cavity B.

According to the ultrasound probe of the modified example, between the clamped member 4 protruded from the tip end side position P1 and the clamped member 4 protruded from the base end side position P2, the operator can change the pair of clamped surfaces to be clamped in one of those two clamped members 4 while clamping the other clamped member 4.

### Modified Example 2 of the Second Embodiment

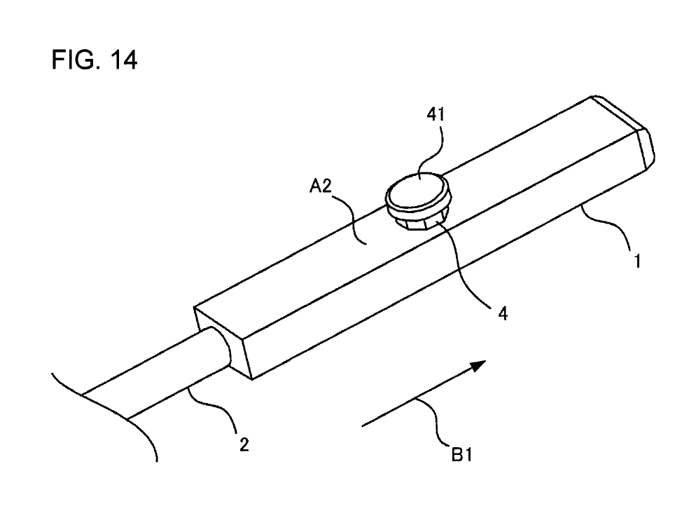

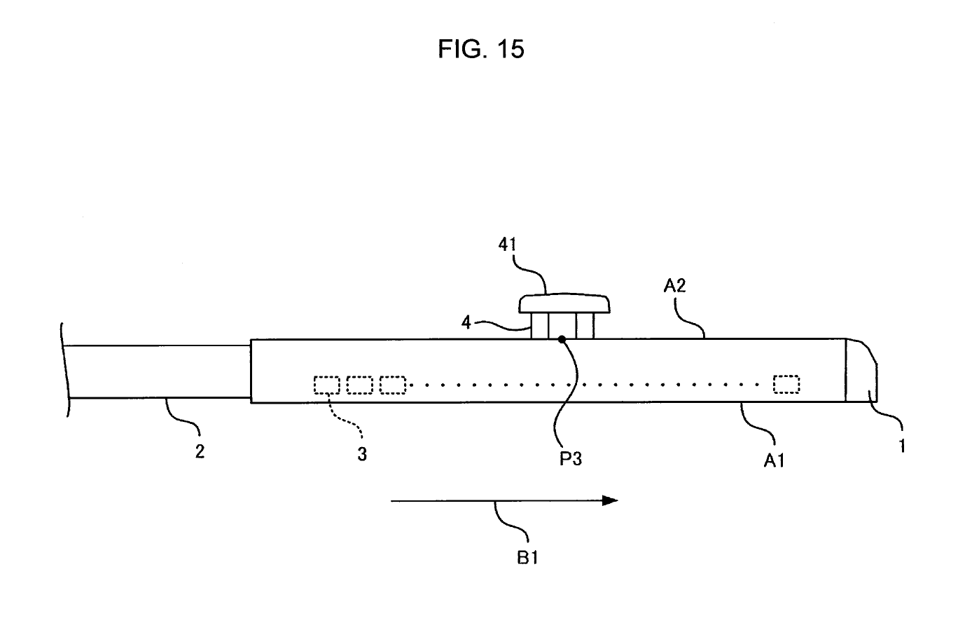

FIG. 14 is a perspective diagram schematically illustrating an ultrasound probe according to a modified example 2 of the second embodiment. FIG. 15 is a side-elevation diagram schematically illustrating the ultrasound probe according to the modified example. The position of the clamped member 4 of the ultrasound probe of the modified example is different from that of the second embodiment. Hereinafter, matters which are different from that of the second embodiment are mainly described.

The clamped member 4 is formed so as to protrude from the central position P3 of the probe head 1. For example, the operator clamps the clamped member 4 protruded from the central position P3 using the forcep 5. Typically, the central position P3 is a position close to the center of the gravity of the probe head 1. Thus, the operator can stably clamp the clamped member 4 protruded from the central position P3 using the forcep 5. For example, when the operator presses the probe head 1 against the subject, such as organs, and the like, the operator can press the probe head 1 against thereto while stably clamping the clamped member 4 protruded from the central position P3.

### Third Embodiment

### [Configuration]

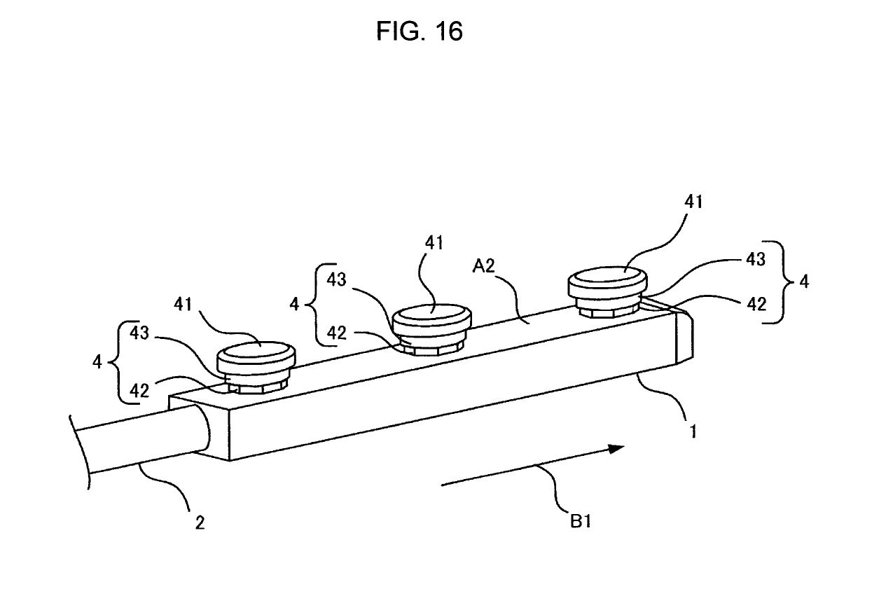

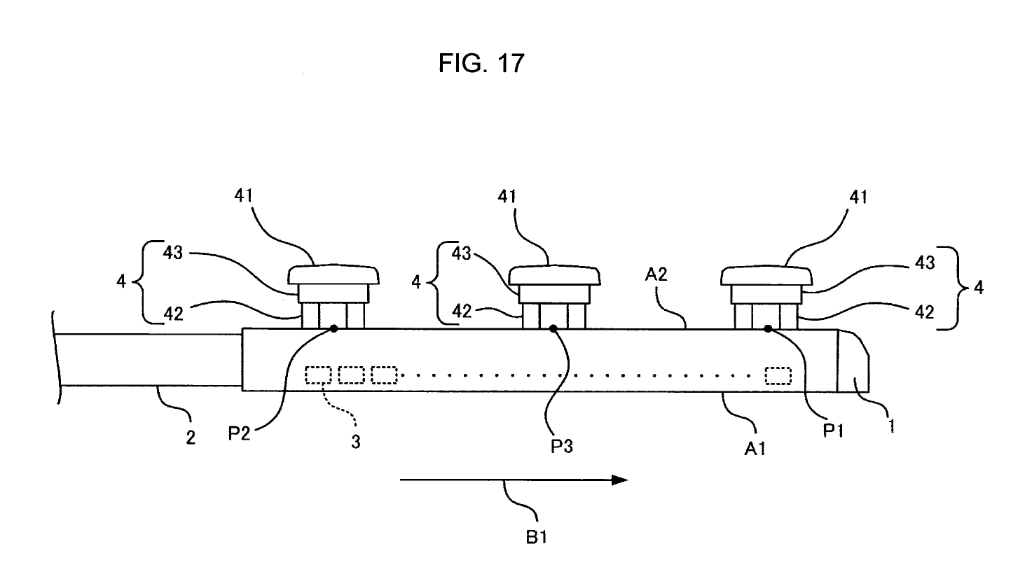

FIG. 16 is a perspective diagram schematically illustrating an ultrasound probe according to a third embodiment. FIG. 17 is a side-elevation diagram schematically illustrating the ultrasound probe of the embodiment. The shape of the clamped member 4 of the ultrasound probe of the embodiment is different from that of the first embodiment and the second embodiment. Hereinafter, matters which are different from that of the first embodiment and the second embodiment are mainly described.

The clamped members 4 of the embodiment are respectively formed so as to protrude from the tip end side position P1, the base end side position P2, and the central position P3 of the probe head 1. Each of the clamped members 4 has a polygonal pillar member 42, and a cylinder member 43.

The polygonal pillar member 42 is formed in a polygonal pillar shape corresponding to the pairs of the clamped surfaces. For example, the polygonal pillar member 42 is formed in a polygonal pillar shape in the similar manner as the first embodiment.

The cylinder member 43 is formed in a cylinder shape. In the cylinder member 43, the diameter of the circle which is the cross-section perpendicular to the axis of the cylinder member 43 is set longer than the length of the diagonal lines of the cross-section perpendicular to the axis of the polygonal pillar member 42. The diameter may be set shorter than the diagonal lines, or equal thereto.

The polygonal pillar member 42 and the cylinder member 43 are arrayed in the protruding direction of the clamped member 4. The polygonal pillar member 42 and the cylinder member 43 are coaxially provided. The axis of the polygonal pillar member 42 and the axis of the cylinder member 43 are provided in parallel with the protruding direction of the clamped member 4. The polygonal pillar member 42 and the cylinder member 43 are arranged from the base end side to the tip end side of the clamped member 4 in the order of the polygonal pillar member 42 and the cylinder member 43. The polygonal pillar member 42 and the cylinder member 43 may be arranged from the base end side to the tip end side of the clamped member 4 in the order of the cylinder member 43 and the polygonal pillar member 42.

### [Effect]

An effect of the ultrasound probe of the embodiment is now described. The clamped member 4 of the ultrasound probe of the embodiment has the polygonal pillar member 42, and the cylinder member 43. The polygonal pillar member 42 is formed in a polygonal pillar shape corresponding to the pairs of the clamped surfaces. The cylinder member 43 is formed in a cylinder shape. The polygonal pillar member 42 and the cylinder member 43 are coaxially arrayed in the protruding direction of the clamped member 4. Thus, when the operator operates the ultrasound probe while clamping the clamped members 4 using two forceps 5, respectively, the operator can control the angle of the ultrasound probe so as to rotate the ultrasound probe while clamping either one or both of the cylinder members 43 of the clamped members 4 using one or both of the forceps 5. The ultrasound probe which can easily control the angle thereof can therefore be provided.

According to at least one of the ultrasound probes of the above-described embodiments, it is possible to improve the operability by the forcep 5.

While certain embodiments have been described, these embodiments have been presented by way of example only, and are not intended to limit the scope of the inventions. Indeed, the novel embodiments described herein may be embodied in a variety of other forms; furthermore, various omissions, substitutions, and changes in the form of the embodiments described herein may be made without departing from the spirit of the inventions. The accompanying claims and their equivalents are intended to cover such forms or modifications as would fall within the scope and spirit of the inventions.

1. An ultrasound probe having a probe head insertable into a body cavity of a subject, wherein
a clamped member including a plurality of pairs of clamped surfaces capable of being clamped by a forcep is formed so as to protrude from a surface which is the opposite side of the surface from where ultrasound waves are emitted, among the surfaces of the probe head.

2. The ultrasound probe according to claim 1, wherein four pairs of the clamped surfaces are formed.

3. The ultrasound probe according to claim 1, wherein
the clamped member includes a polygonal pillar member formed in a polygonal pillar shape having the pair of the clamped surfaces, and a cylinder member formed in a cylinder shape, and
the polygonal pillar member and the cylinder member are coaxially arranged in the protruding direction of the clamped member.

4. The ultrasound probe according to claim 1, wherein the clamped member is provided with a step member which makes the width of a tip end side wider than that of a base end side.

5. The ultrasound probe according to claim 1, wherein the clamped member is formed so as to protrude from a plurality of positions on the surface of the probe head.

6. The ultrasound probe according to claim 5, wherein the clamped member is formed so as to protrude from the plurality of positions arrayed in a longitudinal direction of the probe head.

7. The ultrasound probe according to claim 6, wherein
a plurality of ultrasound transducers of the probe head are arrayed in the longitudinal direction, and
the plurality of positions are arrayed in the same direction as the direction the ultrasound transducers are arrayed.

8. An ultrasound probe capable of being operated by a forcep, wherein
a clamped member capable of being clamped by the forcep is formed so as to protrude from a plurality of positions on a surface different from the surface from where ultrasound waves are emitted, among the surfaces of a probe head.

9. The ultrasound probe according claim 2, wherein
the clamped member includes a polygonal pillar member formed in a polygonal pillar shape having the pair of the clamped surfaces, and a cylinder member formed in a cylinder shape, and
the polygonal pillar member and the cylinder member are coaxially arranged in the protruding direction of the clamped member.
